(19) United States
(12) Patent Application Publication
(10) Pub. No.: US 2021/0267811 A1
(43) Pub. Date: Sep. 2, 2021

(54) BANDAGE DEVICE FOR GLANS AND FORESKIN AND METHODS OF USE

(71) Applicant: David Harper BOONE, Wake Forest, NC (US)

(72) Inventor: David Harper BOONE, Wake Forest, NC (US)

(21) Appl. No.: 17/189,644

(22) Filed: Mar. 2, 2021

Related U.S. Application Data
(60) Provisional application No. 62/983,853, filed on Mar. 2, 2020.

(51) Int. Cl. A61F 13/14

(52) U.S. Cl. CPC A61F 13/148; A61B 17/326

(57) ABSTRACT

A bandage device for the glans and foreskin of a penis. The bandage device includes a securing member that extends from a first end to a second end and a pad. The pad is shaped to surround the glans of the penis and the securing member can be placed around the glans to both secure the pad and prevent the foreskin from fully extending. The securing member may preferably have elastic properties and the pad is composed of absorptive materials. In use, the foreskin is pulled back, the pad is placed over the glans, the securing member is looped around the penis, secured, and the foreskin is then released. Medication may be applied to the pad prior to application of the bandage device.

### CROSS-REFERENCE TO RELATED APPLICATIONS

To the full extent permitted by law, the present United States Non-Provisional Patent Application hereby claims priority to and the full benefit of, United States Provisional Application entitled “BANDAGE DEVICE FOR GLANS AND FORESKIN,” having assigned Ser. No. 62/983,853, filed on Mar. 2, 2020, which is incorporated herein by reference in its entirety.

### FEDERALLY SPONSORED RESEARCH OR DEVELOPMENT

None

### PARTIES TO A JOINT RESEARCH AGREEMENT

None

### REFERENCE TO A SEQUENCE LISTING

None

### BACKGROUND OF THE DISCLOSURE

### Technical Field of the Disclosure

The instant disclosure relates to bandages, namely bandaging systems. More particularly, the instant disclosure relates to a two-part system for maintaining dryness, preventing microorganismal growth, and promoting the healing of an anatomical extremity.

### Description of the Related Art

The human foreskin is a double-layered fold of smooth muscle tissue, blood vessels, neurons, skin, and mucous membrane part of the penis that covers and protects the glans penis and the urinary meatus. The foreskin is mobile, fairly stretchable, and acts as a natural lubricant during movement and coitus. It is also described as the prepuce, a technically broader term that also includes the clitoral hood in women, to which the foreskin is embryonically homologous. The foreskin of adults is typically retractable over the glans. Coverage of the glans in a flaccid and erect state varies depending on foreskin length. The foreskin is attached to the glans at birth and is generally not retractable in infancy. The foreskin may become subject to a number of pathological conditions. Most conditions are rare and are easily treated. In some cases, particularly with chronic conditions, treatment may include circumcision, a procedure where the foreskin is partially or completely removed. In males, circumcision may also occur at infancy, based on the customs, religion, or tradition of an infant's parents, it may occur later due to a preference of the adult male or due to one of many chronic conditions, or it may never occur and the male may perish with an intact foreskin.

The outside of the foreskin is a continuation of the skin on the shaft of the penis, but the inner foreskin is a mucous membrane like the inside of the eyelid or the mouth. The mucocutaneous zone occurs where the outer and inner foreskin meet. Like the eyelid, the foreskin is free to move after it separates from the glans, which usually occurs before or during puberty. The foreskin is attached to the glans by a frenulum, a highly vascularized tissue of the penis. The frenulum forms the interface between the outer and inner foreskin layers, and when the penis is not erect, it tightens to narrow the foreskin opening.

The human foreskin contains a sheath of muscle tissue just below the skin, most of which is contained in the foreskin. Elastic fibers are contained in the dartos fascia, which form a whorl at the tip of the foreskin. The whorl of fibers acts as a sphincter in infants, which opens to allow the passage of urine, but closes to protect the glans penis from foreign matter and contaminants. The dartos fascia is sensitive to temperature and expands and contracts with temperature changes. The dartos fascia is only loosely connected with the underlying tissue so it provides the skin mobility and elasticity of the penile skin. Langerhans cells are immature dendritic cells that are found in all areas of the penile epithelium, but are most superficial in the inner surface of the foreskin.

Anatomically, the foreskin may serve a variety of other purposes and functions, some of which may be disputed and/or otherwise controversial. However, many human medical conditions implicating the foreskin do exist, and due to the unique anatomical nature of the skin combined with the penis's size modulating capabilities, these medical conditions may be exacerbated by the involuntary nature of the foreskin to cover the flaccid penis. Notwithstanding its function, importance, or other reasons a male adult may wish remain intact, an intact male may face some of these serious, though rare, conditions. First, the foreskin can be involved in balanitis, phimosis, sexually transmitted infection and penile cancer. Another conditions, frenulum breve, is a frenulum that is insufficiently long to allow the foreskin to fully retract, which may lead to discomfort during intercourse. Phimosis is a condition where the foreskin of an adult cannot be retracted properly. Phimosis can be treated by using topical steroid ointments and using lubricants during sex. Circumcision may be indicated for severe cases. A condition called paraphimosis may occur if a tight foreskin becomes trapped behind the glans and swells as a restrictive ring. This can cut off the blood supply, resulting in ischemia of the glans penis. Posthitis is a bacterial or fungal infection that causes inflammation of the foreskin. Some birth defects of the foreskin can occur, though all of them are rare. In aposthia there is no foreskin at birth, in micropathia the foreskin does not cover the glans, and in macroposthia, also called and congenital megaprepuce, the foreskin extends well past the end of the glans. Though HIV is not inherently or exclusively the domain of intact males, it has been found that larger foreskins place uncircumcised men at an increased risk for HIV infection most likely due to the larger surface area of inner foreskin and the high concentration of Langerhans cells. Finally, medical and urinary conditions such as incontinence, prostate issues, or other issues causing the involuntary leaking of urine from the bladder through the penis, may have different effects between circumcised and intact males.

Circumcision is the removal of the foreskin, either partially or completely. In the most common procedure, the foreskin is opened, adhesions are removed, and the foreskin is separated from the glans. After that, a circumcision device may be placed, and then the foreskin is cut off. Topical or locally injected anesthesia is generally used to reduce pain and physiologic stress. The procedure is most often an elective surgery performed on babies and children for religious or cultural reasons. Approximately one-third of adult males worldwide are circumcised. Medically, circumcision is a treatment option for problematic cases of phimosis and balanoposthitis that do not resolve with other treatments, and for chronic urinary tract infections (UTIs). As mentioned above, for newborns, it may be done for religious requirements or personal preferences surrounding hygiene and aesthetics. Circumcision may also be performed on children or adults to treat the above conditions including phimosis, balanitis, or to prevent transmission of sexually transmitted infections. As of today, no successful technique to reconstruct a circumcised foreskin had been published. Some circumcised adult males have used weights to stretch the skin of the penis to regrow a foreskin, but the resulting tissue does cover the glans but does not replicate the features of a foreskin. Additionally, the practice of infant male circumcision has maintained a state of controversy throughout the world. The positions of the world's major medical organizations range from a belief that elective circumcision of babies and children carries significant risks and offers no medical benefits to a belief that the procedure has a modest health benefit that outweighs small risks. No major medical organization recommends circumcising all males, and no major medical organization recommends banning the procedure, though organizations do exist to advocate for the latter. Since the process of circumcision is currently medically irreversible, and since all surgical interventions carry an associated risk, many intact males wish to remain so.

In cases where a male adult has a condition where urine or other fluid excretions prevent the glans area of the penis from fully drying, bacterial and fungal growth may become a problem. Various products currently on the market attempt to resolve this problem by encouraging and maintaining dryness through absorptive materials. These include adult diapers and male-sized underwear liners or pads. They may also include gauze wrapping or other techniques which may be adapted for use for this condition. With regard to diapers, many adult males may suffer embarrassment, anxiety, frustration, or other physical and/or emotional tolls from the use of adult diapers. While pads or underwear liners may not cause the same extent of physical and/or emotional tolls, they also suffer from the detriment of fluid escaping around the pad, therefore causing further embarrassment should the fluid be noticeable to third parties.

Therefore, it is readily apparent that there is a recognized unmet need for an apparatus for use on the male glans and foreskin to prevent the buildup of moisture, to facilitate the maintenance of dryness, and to promote cleanliness and healing of the area. The instant disclosure is designed to address this need through an apparatus and methods of use, which includes the bandage disclosed herein while addressing at least some of the aspects of the problems discussed above.

### SUMMARY

Briefly described, in a possibly preferred embodiment, the present disclosure overcomes the above-mentioned disadvantages and meets the recognized need for such a bandage device by providing a 2-part bandage device that in combination assist in the pulling back of the foreskin in uncircumcised, intact individuals while simultaneously absorbing fluid and/or applying medication and/or ointment. The bandage device's two parts may be used individually or in combination to assist in preventing moisture buildup, facilitate air drying of the glans area, and providing an absorptive surface by which medication or ointment may be applied and retained on the glans area.

More specifically, the example embodiments of the present bandage device may comprise a securing member and a pad. The securing member, or foreskin holding device/bandage, may be a narrow and long band having elastic properties. It may further comprise a hook portion and loop portion to allow the application and securing in an easy manner. It may further be manufactured or formed in various lengths to accommodate a range of individuals having a range in circumference of penises. Preferably, the length of the securing member may accommodate a patient in that the length of the securing member may approximate the circumference of the patient's penis. Alternatively, a length of the hook or loop portions of the securing member may extend along a large portion of one side of the securing member so as to accommodate a range of patient penis circumferences. The pad may be formed from an absorptive material. The size of the pad may vary depending on volume of expected liquid (urine), volume of intended medication/ointment, and size of the patient penis.

In an exemplary embodiment, the bandage device for glans and foreskin may operably engage the pad with the securing member through a variety of mechanisms. The simplest means of operable engagement may be simple friction—simply placing the pad over the end of the patient penis, and securing the securing member to the pad and penis. Additional security may include adhesive on the pad element, additional hook and loop elements, or other methods of securing two fabric materials to each other known to those skilled in the art.

In a further exemplary embodiment, the securing member may be used alone or in combination with the pad. Alone, the securing member may provide the benefit of allowing a patient having irritation, infection, or other discomfort, the ability to pull back and maintain a pulled back foreskin. This may allow the patient to air dry the glans. In combination, the securing member and the pad may further encourage drying and facilitate healing. The pad may offer the additional benefit of avoiding moisture leaking from the penis from being visibly detectable through clothing.

In select embodiments, the bandage device for glans and foreskin may have a stretchable securing member and/or a securing member having elastic properties. This may offer the additional benefit of allowing comfortable securing ability to a patient penis while simultaneously allowing the device to stretch to accommodate fluctuation of penis size during normal penis expansion and contraction. Additionally, it may offer the additional benefit of preventing potential injury or discomfort should a patient wearing the bandage device experience an erection of the penis.

In other embodiments, the pad may have a specialized shape so as to accommodate the normal anatomical shape of the glans area of the penis. These shapes may generally include semi-circles, trapezoid, triangle, circle, spade, or other shapes one skilled in the art would know may offer benefits for the intended purpose of absorbing moisture and/or retaining medication/ointment. Additionally, the pad may be constructed from a variety of known absorptive materials or textiles.

A feature of the bandage device may be the ease of application. A physician, medical professional, or patient alike may be able to easily attach the bandage device to the penis of the patient. Contributing to this ease of application may be a two-step or two-part application process. In a potentially preferred embodiment of both the bandage device and its method of use, application may be performed in a matter of seconds. This may be especially important, given the likelihood that an individual applying such a bandage may want to be discrete during application.

Another feature of the bandage device may be the affordability and disposability of the bandage device. Since cleanliness and comfortability of the area may be important to the area which the bandage device may be applied, multiple attempts may be necessary. Additionally, it may be necessary for a patient to regularly change the bandage device. Affordability and disposability may be important to patients wishing to keep this area clean, dry, and comfortable.

In use, a potentially preferred method of use of the bandage device of the disclosure may begin by providing the 2-part bandage device to a patient experiencing a symptom which the bandage device may assist. Then the patient, or a treating healthcare provider, may pull back the foreskin of the patient. A preparation may be placed, inserted, and/or spread into an opening of the pad opening in an optional step. The preparations may include but are not limited to medicine, lotion, ointment, drying agents, skin protectants, petroleum jellies, the like and/or combinations thereof. An optional adhesive area of the pad, which may have a backing, may optionally be exposed or adhesive may optionally be applied and the pad may be placed over the glans of the penis via the opening of the pad. Finally, the patient, or a healthcare provider providing care to the patient, may wrap the securing member around the penis below the glans. In cases where some or all the optional steps are included, the securing member may also be wrapped around and/or over the pad. In either case, this final step includes the sub-step of securing the securing member around the penis.

These and other features of the bandage device for glans and foreskin and methods of use will become more apparent to one skilled in the art from the prior Summary and following Brief Description of the Drawings, Detailed Description of exemplary embodiments thereof, and Claims when read in light of the accompanying Drawings or Figures.

### BRIEF DESCRIPTION OF THE DRAWINGS

The bandage device for glans and foreskin and methods of use will be better understood by reading the Detailed Description with reference to the accompanying drawings, which are not necessarily drawn to scale, and in which like reference numerals denote similar structure and refer to like elements throughout, and in which:

FIG. 1 is a bottom view of the securing member of the bandage.

FIG. 2 is a top view of the securing member of the bandage.

FIG. 3 is a top view of the pad of one embodiment of the bandage.

FIG. 4 is a sectional view of the pad of the bandage.

FIG. 5 is a perspective view of a penis having the pad and securing member of the bandage secured thereon.

FIG. 6 is a perspective view of the penis having the pad and securing member of the bandage secured thereon.

FIG. 7 is a flow chart of an exemplary methods of use of the bandage of the disclosure.

It is to be noted that the drawings presented are intended solely for the purpose of illustration and that they are, therefore, neither desired nor intended to limit the disclosure to any or all of the exact details of construction shown, except insofar as they may be deemed essential to the claimed disclosure.

### DETAILED DESCRIPTION

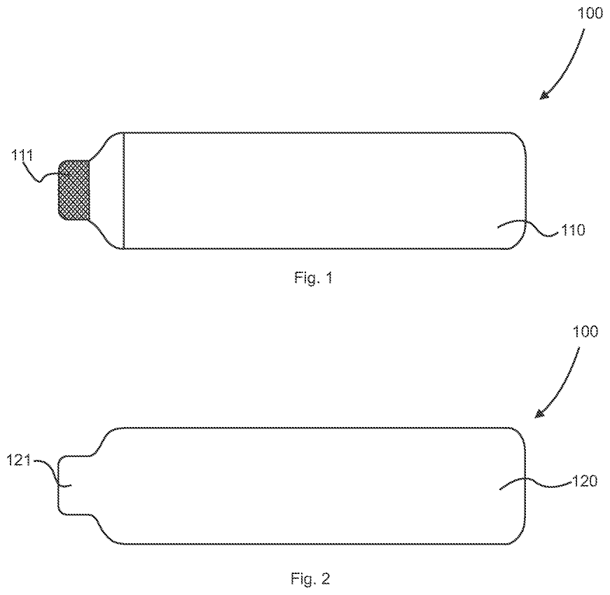

In describing the exemplary embodiments of the present disclosure, as illustrated in FIGS. 1-7, specific terminology is employed for the sake of clarity. The present disclosure, however, is not intended to be limited to the specific terminology so selected, and it is to be understood that each specific element includes all technical equivalents that operate in a similar manner to accomplish similar functions. Embodiments of the claims may, however, be embodied in many different forms and should not be construed to be limited to the embodiments set forth herein. The examples set forth herein are non-limiting examples, and are merely examples among other possible examples. It should be noted that the term male may be used herein as a descriptor for a human being having a penis. The term male, as used herein, shall mean an individual having a penis, or an individual having the male sex and corresponding intact male genitalia.

Referring now to FIGS. 1-6 by way of example, and not limitation, therein are illustrated example embodiments of the bandage for glans and foreskin having securing member 100 and pad 200. The bandage for glans and foreskin may be referred herein as just the bandage. Securing member 100 in combination with pad 200 may be referred herein as just the bandage. The bandage may be used on penis P. Although penis P is shown in the drawings as an adult visibly erect penis, the disclosure is not so limited, and the bandage device of the disclosure may be used on any human extremity or the like, including, but not limited to: flaccid penises, the penises of children, fingers, toes, noses, ears, the like, and/or combinations thereof

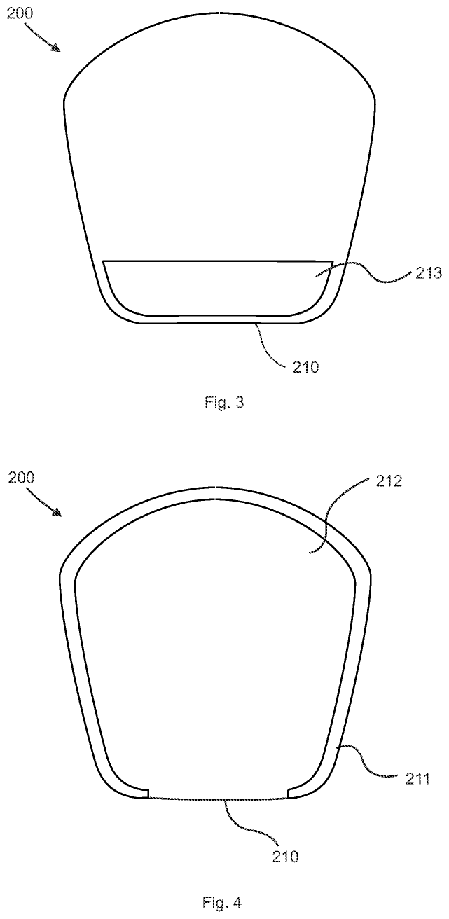

In one embodiment, the bandage may provide the following elements: securing member 100 (see FIGS. 1, 2, 5, and 6) and pad 200 (see FIGS. 3, 4, 5, and 6). Further enhancing its capabilities, the bandage may secure to penis P by first placing pad 200 over penis P, then pulling back foreskin F and looping securing member 100 around pad 200. The bandage may be applied and worn for a small or large amount of time and may be composed of materials suitable for disposal after use. The method of replacing the bandage may be required to be performed regularly, as either pad 200 becomes filled with liquid or for hygienic purposes.

Referring now more specifically to FIG. 1, illustrated therein is an example of a bottom side of securing member 100 of the bandage device of the disclosure. Starting at the left, illustrated therein FIG. 1 is securing mechanism 111 and bottom surface 110. Securing member 100 and bottom surface 110 may form a specific length from a first end to a second end of securing member 100. One skilled in the art would understand that this specific length may be best understood to be substantially similar, or slightly greater, to the diameter of a penis of a patient. Securing mechanism 111 may implicate a variety of known means to secure a material to another material. Though the disclosure posits hook-and-loop-type fasteners, which may be comprised of hook fasteners intended to be engaged operably with loop fasteners, to be a potentially preferred embodiment of securing mechanism 111 to top surface 120, many variations of potentially useful fastening techniques are contemplated herein. By way of example and not limitation, other variations of fastening techniques to adhere securing mechanism 111 to top surface 120 may include molded hook (e.g. EASY TAPE® molded hook), J-hooks, palm tree hooks, mushroom hooks, magnets, buttons, adhesive, the like, and/or combinations thereof. Securing member 100 may be substantially constructed of materials known by those skilled in the art of bandage manufacture. These materials may include, but are not limited to, plastics, cloth, woven fabrics, non-woven fabrics, synthetics, composites, elastics, the like and/or combinations thereof. In a potentially preferred embodiment of securing member 100, it may be substantially constructed of a material capable of expanding and contracting to accommodate the normal fluctuations in size of the human penis. Bottom surface 110 may additionally include a thickness, or padding. Ideally, padding would be of a suitable material and thickness to encourage comfort, expansion, and hypoallergenic properties.

Referring now more specifically to FIG. 2, illustrated therein is an example of a top view of securing member 100 of the bandage device of the disclosure. Beginning at the left, illustrated therein FIG. 2 is tab portion 121 followed by top surface 120. Top surface 120 may operably engage securing mechanism 111. Top surface 120 may further share a length with bottom surface 110. Top surface 120 may operably engage securing mechanism 111 along its entire length, or alternatively, may only operably engage securing mechanism 111 in a specific region, depending on the desired circumference, based on patient penis circumference. Securing mechanism 111 may implicate a variety of known means to secure a material to another material. Though the disclosure posits hook-and-loop-type fasteners to be a potentially preferred embodiment of securing mechanism 111 to top surface 120, many variations of potentially useful fastening techniques are contemplated herein. By way of example and not limitation, other variations of fastening techniques to adhere securing mechanism 111 to top surface 120 may include molded hook (e.g. EASY TAPE® molded hook), J-hooks, palm tree hooks, mushroom hooks, magnets, buttons, adhesive, the like, and/or combinations thereof. Securing member 100 may be substantially constructed of materials known by those skilled in the art of bandage manufacture. These materials may include, but are not limited to, plastics, cloth, woven fabrics, non-woven fabrics, synthetics, composites, elastics, the like and/or combinations thereof. In a potentially preferred embodiment of securing member 100, it may be substantially constructed of a material capable of expanding and contracting to accommodate the normal fluctuations in size of the human penis.

Referring now more specifically to FIG. 3, illustrated therein is a top view of pad 200 of one embodiment of the bandage device of the disclosure. Pad 200, in a potentially preferred embodiment may utilize adhesive area 213 to operably engage with securing member 100. Adhesive area 213 may utilize a peel-away backing so as to allow for protection of adhesive prior to application or use. This peel-away aspect of adhesive area 213 may provide the further benefit of preventing adhesion to dust, dirt, the person applying the bandage device, or anything else, and therefore may encourage or facilitate cleanliness of pad 200 and adhesive area 213, or adhesive layer. At the bottom of pad 200, opening 210 may allow entry of the glans of the patient penis into pad 200. Bottom view (not pictured) may be substantially identical to top view of pad 200. Alternatively, one side, but not the other, may have adhesive area 213. This may decrease costs of manufacture as well as ease of application of pad 200, securing member 100, and the bandage device of the disclosure. Alternatively, inclusion of adhesive area 213 on both sides of pad 200 may increase adhesion properties of pad 200. It is contemplated herein that medicine, lotion, ointment, drying agents, skin protectants, petroleum jellies, the like and/or combinations thereof may be inserted into opening 210 prior to, during, or after application of pad 200. Pad 200, in the exemplary embodiment of FIG. 3 may be understood to be spade-shaped or rounded pentagonal-shaped, but the disclosure is not so limited. Other exemplary shapes include but are not limited to: circular, semi-circular (or portion of a circle), triangular, quadrilateral, rectangular, square, hexagonal, octagonal, nine or more sided polygonal, rounded variations thereof, the like, and/or combinations thereof. While it is understood that pad 200 may lie flat in packaging, as described further below, it may expand to accommodate the end of a patient penis. In a potentially preferred embodiment of pad 200, it may be constructed of woven gauze or non-woven gauze. Other materials that may be suitable for use as a wound dressing, materials that are absorptive and capable of sterility, may be utilized for construction and/or manufacture of pad 200. Adhesive area 213 may be composed of any variety of adhesives known by those skilled in the art of bandage manufacture including but not limited to: methacrylate, epoxy diacrylate, the like and/or combinations thereof. As mentioned above, adhesive area 213 may be absent in alternate embodiments, allowing for friction alone to secure pad 200 via securing member 100.

Referring now more specifically to FIG. 4, illustrated therein is a sectional view of pad 200 of the bandage device of the disclosure. Pad 200 may include opening 210, internal region 212, and have thickness 211. Increasing the size of thickness 211 may increase the moisture absorbing capabilities of pad 200. Internal region 212 may be best understood as an area to house an end of a patient penis, or the glans area. It also may be understood to be an area ideal for certain preparations to encourage healing and/or drying of the glans. It is contemplated herein that those preparations may include but are not limited to medicine, lotion, ointment, drying agents, skin protectants, petroleum jellies, the like and/or combinations thereof may be inserted into opening 210 prior to, during, or after application of pad 200. Pad 200, in the exemplary embodiment of FIG. 3 may be understood to be spade-shaped or rounded pentagonal-shaped, but the disclosure is not so limited. Other exemplary shapes include but are not limited to: circular, semi-circular (or portion of a circle), triangular, quadrilateral, rectangular, square, hexagonal, octagonal, nine or more sided polygonal, rounded variations thereof, the like, and/or combinations thereof. While it is understood that pad 200 may lie flat in packaging, as described further below, it may expand to accommodate the end of a patient penis. In a potentially preferred embodiment of pad 200, it may be constructed of woven gauze or non-woven gauze. Other materials that may be suitable for use as a wound dressing, materials that are absorptive and capable of sterility, may be utilized for construction and/or manufacture of pad 200. Pad 200, and internal region 212 particularly, in a potentially preferred embodiment, may include a pre-medicated area or may be constructed, manufactured, or modified to include a layer of medication within internal region 212 on its surface, which may be understood to be a medicated layer. Thickness 211 and pad 200 may be best understood as a substrate layer having the capability of absorbing moisture.

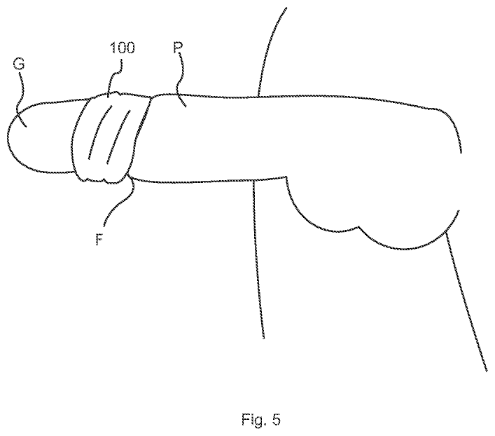

Referring now to FIG. 5 by way of example, and not limitation, therein is illustrated a perspective view of penis P having only securing member 100 of the bandage device of the disclosure secured thereon. Penis P, shown herein as potentially erect, may also be in a flaccid or semi-erect state during application of securing member 100. Foreskin F may be pulled back during application, exposing glans G, securing member 100 wrapped around penis P, secured, and the foreskin is then released. Application of securing member 100 alone may encourage the drying or air drying of glans G. Securing member 100 may be substantially constructed of materials known by those skilled in the art of bandage manufacture. These materials may include, but are not limited to, plastics, cloth, woven fabrics, non-woven fabrics, synthetics, composites, elastics, the like and/or combinations thereof. In a potentially preferred embodiment of securing member 100, it may be substantially constructed of a material capable of expanding and contracting to accommodate the normal fluctuations in size of the human penis.

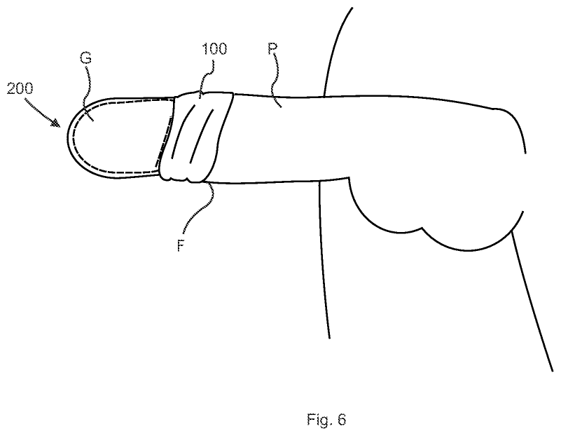

Referring now to FIG. 6 by way of example, and not limitation, therein is illustrated a perspective view of penis P having both pad 200 and securing member 100 of the bandage device of the disclosure secured thereon. Penis P, shown herein as potentially erect, may also be in a flaccid or semi-erect state during application of pad 200 and securing member 100. Foreskin F may be pulled back during application, exposing glans G, placing pad 200 on glans G through opening 210, securing member 100 wrapped around pad 200 and penis P, securing securing member 100, and the foreskin is then released. Application of securing member 100 in combination with pad 200 may encourage the drying of glans G or, additionally, may sustain dryness should leaking occur from the urethra of penis P or from secretions from glans G. Securing member 100 may be substantially constructed of materials known by those skilled in the art of bandage manufacture. These materials may include, but are not limited to, plastics, cloth, woven fabrics, non-woven fabrics, synthetics, composites, elastics, the like and/or combinations thereof. In a potentially preferred embodiment of securing member 100, it may be substantially constructed of a material capable of expanding and contracting to accommodate the normal fluctuations in size of the human penis. In a potentially preferred embodiment of pad 200, it may be constructed of woven gauze or non-woven gauze. Other materials that may be suitable for use as a wound dressing, materials that are absorptive and capable of sterility, may be utilized for construction and/or manufacture of pad 200. Additionally, to encourage drying, sustain drying, or encourage healing, preparations may be inserted into opening 210 prior to placing glans G into pad 200. It is contemplated herein that those preparations may include but are not limited to medicine, lotion, ointment, drying agents, skin protectants, petroleum jellies, the like and/or combinations thereof may be inserted into opening 210 prior to, during, or after application of pad 200. In an alternative embodiment of the bandage device of the disclosure, adhesive area 213 may instead reside in internal region 212. In such an embodiment, pad 200 may be applied after securing securing member 100 to penis P.

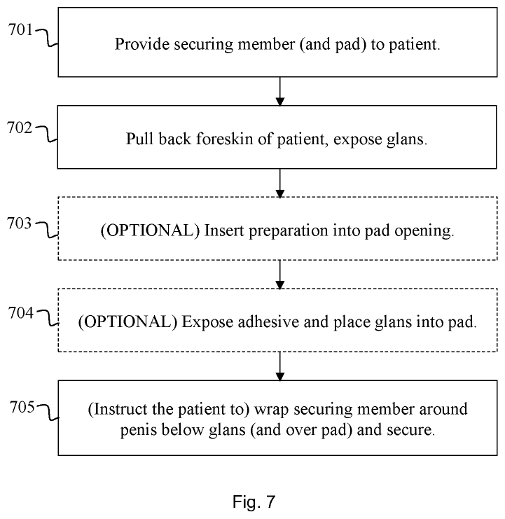

Referring now more specifically to FIG. 7, there is illustrated a flow diagram of a method of application or use of the bandage device of the disclosure. At first method step 701, the bandage device of the disclosure may be provided to the patient, which may include at least securing member 100 but may also include pad 200. At second method step 702, the patient, or a treating healthcare provider, may pull back the foreskin of the patient. At third method step 703, which may be optional, a preparation may be placed, inserted, and/or spread into pad opening 210. It is contemplated herein that those preparations may include but are not limited to medicine, lotion, ointment, drying agents, skin protectants, petroleum jellies, the like and/or combinations thereof. At fourth method step 704, each sub-step of which also may be optional, adhesive area 213 of pad 200, which may have a removable backing, may be exposed or adhesive may be applied and pad 200 may be placed over glans G of penis P via opening 210. Finally, at fifth method step 705, the patient, or a healthcare provider providing care to the patient, may wrap securing member 100 around penis P below glans G. In cases where some or all of third method step 703 and some or all of fourth method step 704 are included, securing member 100 may also be wrapped around and/or over pad 200. In either case, fifth method step 705 includes the sub-step of securing securing member 705.

It is contemplated herein that certain benefits, including increased security, increased longevity, increased comfortability, and other benefits may be conferred should pad 200 extend past, toward the patient, in comparison to securing member 100. In other words, pad 200 may be of such length that it can be observed over glans G, but also on the other side of securing member 100, when the bandage device of the disclosure is fully applied to penis P. It is also further contemplated that pad 200 may be provided in one or more sizes, or may have large or small thickness 211. Thickness 211 is understood to increase or decrease absorptive properties as it increases or decreases in size, respectively. If the bandage of the disclosure is intended to provide moisture absorbing benefits to the patient, an increase in thickness 211 may be indicated. If only medicating preparation delivery is required, thickness 211 may be limited or not indicated as important to the retention and delivery of a preparation to glans G. On external surface of pad 200 may reside a waterproof or non-absorptive layer thereby offering the capability of sealing in moisture as pad 200 fills with liquid. Additionally, pad 200 and securing member 100 may each contain a color-changing feature having the ability to change to an indicated color when moist. While primary benefits and uses of the bandage device of the disclosure may be to provide drying, moisture absorbing, and preparation delivery to the glans of an intact or uncircumcised post-pubescent male, the disclosure is not so limited. Other uses of the bandage device of the disclosure may include but are not limited to: (i) providing a bandage to a human extremity (e.g. finger, toe, ear, or nose), (ii) providing similar benefits to circumcised post-pubescent males, (iii) providing post-circumcision care to post-pubescent males, (iv) providing similar benefits to intact or circumcised males experiencing one of the symptoms herein described, regardless of pre- or post-pubescence (i.e. regardless of age or presence of foreskin F on penis P), and (v) post-operative care for the phallus of a female-to-male transexual/transgender person.

The foregoing description and drawings comprise illustrative embodiments. Having thus described exemplary embodiments, it should be noted by those skilled in the art that the within disclosures are exemplary only, and that various other alternatives, adaptations, and modifications may be made within the scope of the present disclosure. Merely listing or numbering the steps of a method in a certain order does not constitute any limitation on the order of the steps of that method. Many modifications and other embodiments will come to mind to one skilled in the art to which this disclosure pertains having the benefit of the teachings presented in the foregoing descriptions and the associated drawings. Although specific terms may be employed herein, they are used in a generic and descriptive sense only and not for purposes of limitation. Accordingly, the present disclosure is not limited to the specific embodiments illustrated herein, but is limited only by the following claims.

What is claimed is:

1. A bandage device for a patient with a penis having a glans and a foreskin, the bandage device comprising:
a securing member extending from a first end to a second end, the securing member having a top surface and a bottom surface, wherein at least a portion of the bottom surface is configured to engage at least a portion of the top surface of the securing member; and
a pad configured to envelop at least a portion of the glans of the penis, the pad including a substrate layer configured to absorb moisture;
wherein the pad is configured to operably engage with the securing member, thereby securing the pad to the glans of the penis.

2. The bandage device of claim 1, wherein said portion of the bottom surface comprises hook fasteners and wherein said portion of the top surface comprises loop fasteners.

3. The bandage device of claim 1, wherein said pad further comprises a medicated layer.

4. The bandage device of claim 1, wherein said pad further comprises an adhesive layer.

5. The bandage device of claim 4, wherein said adhesive layer has a removable backing.

6. The bandage device of claim 1, wherein the pad operably engages the securing member by wrapping the securing member around the pad and connecting the top surface to the bottom surface of the securing member.

7. The bandage device of claim 6, wherein the top surface and the bottom surface of the securing member engage one another via an interlocking material and wherein the bottom surface further comprises a padding.

8. The bandage device of claim 7, wherein the interlocking material of the top surface and the bottom surface of the securing member is a hook and loop fastener.

9. The bandage device of claim 8, wherein said pad further comprises an adhesive layer on an at least one side of an exterior side of said pad, and said pad operably engages said securing member using said adhesive layer.

10. The bandage device of claim 1, wherein a distance between said first end and said second end of said securing member approximates a circumference of the penis.

11. A bandage device comprising:
a securing member extending from a first end to a second end, the securing member having a top surface and a bottom surface, wherein at least a portion of the bottom surface is configured to engage at least a portion of the top surface of the securing member; and
a pad configured to envelop at least a portion of an end of a human extremity, the pad including a substrate layer configured to absorb moisture;
wherein the pad is configured to operably engage with the securing member, thereby securing the pad to the human extremity.

12. A method of absorbing moisture from a penis of a patient, the patient and the penis having a foreskin, the method comprising:
providing a bandage device, the bandage device comprising:
a securing member extending from a first end to a second end, the securing member having a top surface and a bottom surface, wherein at least a portion of the bottom surface is configured to engage at least a portion of the top surface of the securing member; and
a pad having an opening configured to envelop at least a portion of the glans of the penis, the pad including a substrate layer configured to absorb moisture;
pulling back the foreskin of the penis to expose a glans;
placing the pad over said glans;
securing the pad to the penis by wrapping the securing member around the pad and engaging the top surface with the bottom surface.

13. The method of claim 12, further comprising a step of placing a preparation into the pad via the opening.

14. The method of claim 13, wherein the preparation is a medication.

15. The method of claim 12, wherein the pad further comprises a medicated layer with an interior portion of the pad.

16. The method of claim 12, wherein the top surface and the bottom surface of the securing member engage one another via an interlocking material

17. The method of claim 16, wherein the bottom surface of the securing member further comprises a padding.

18. The method of claim 16, wherein the interlocking material is a hook and loop fastener.

19. The method of claim 12, wherein the pad further comprises an adhesive layer on an at least one side of an exterior side of the pad, and the pad operably engages the securing member using the adhesive layer.

20. The method of claim 19, further comprising a protective and removable backing placed over the adhesive layer of the pad, and further includes a step of peeling the protective and removable backing prior to the step of securing the pad to the penis by wrapping the securing member around the pad and engaging the top surface with the bottom surface, thereby more securely securing the pad to the glans.
